(19) United States
(12) Patent Application Publication
(10) Pub. No.: US 2007/0099187 A1
(43) Pub. Date: May 3, 2007

(54) Nucleic acid array

(75) Inventor: Mamoru Tsukada, Kanagawa-ken (JP)

Correspondence Address: FITZPATRICK CELLA HARPER & SCINTO, 30 ROCKEFELLER PLAZA, NEW YORK, NY, 10112, US

(73) Assignee: Canon Kabushiki Kaisha, Tokyo (JP)

(21) Appl. No.: 10/556,508

(22) PCT Filed: May 13, 2004
(86) PCT No.: PCT/JP04/06797

§ 371 Date: Oct. 26, 2006

(30) Foreign Application Priority Data
May 13, 2003 (JP) 2003-135792

(51) Int. Cl. C12Q 1/68; C07H 21/04

(52) U.S. Cl. 435/6; 536/24.3

(57) ABSTRACT

A nucleic acid array is provided that can efficiently perform analysis, and further, can estimate the number of expressions of amino acid mutations for all cases by use of a smaller number of arrays. The nucleic acid array has a plurality of oligonucleotides immobilized on a substrate, wherein the oligonucleotide has a sequence site that forms base pairs with at least two bases of the four bases (A, T, C, G or U) or has a sequence site that does not form a base pair with any of the four bases.

### TECHNICAL FIELD

The present invention relates to a so-called nucleic acid array for detecting a target nucleic acid, specifically for investigation on genomic diversity, genetic polymorphisms, SNPs and the like, or a mutation or mismatched base pair, and in particular expression of mRNA and the like.

### BACKGROUND ART

Conventionally, methods for analyzing an expression amount of mRNA are not limited to the sequence in particular. Various methods exist including methods that use microarrays in which cDNA fragments acquired through past technological research are immobilized, methods that utilize EST and STS in databases, and methods that are not disclosed to users at all.

As a method that utilizes the microarray method for analyzing an amount of a target nucleic acid in a mixture of two types of nucleic acids derived from two kinds of samples, a method is reported in U.S. Pat. No. 5,800,992 in which a fluorescent label is used as a marker.

However, when mRNA is considered in terms of the corresponding amino acids, since there are 1 to 6 possible triplets of bases corresponding to one amino acid, analysis among individuals is further complicated.

### DISCLOSURE OF THE INVENTION

It is, therefore, an object of the present invention is to provide a nucleic acid array that can perform analysis efficiently, and further, can estimate the number of expressions for all cases of amino acid mutation by use of a smaller number of arrays.

The present inventors have focused on the 1st and the 2nd bases of triplets corresponding to respective amino acids and found that by determining these two bases, the combination of bases representing one amino acid can be limited to one or two possibilities. The sole purpose of the 3rd base of a triplet in this case is to regulate the length of strand with a hybridizing counterpart. Therefore, in the present invention, an oligonucleotide is designed to have a sequence site that forms base pairs with at least two bases of the four bases (A, T, C, G or U) or to have a sequence site that does not form a base pair with any of the above bases. In order to represent only three amino acids in mRNA, the total number of possibilities will be 6×6×6=216 in theory in the case of the maximum number of combinations. In contrast, by using as the sequence site an abasic sugar-phosphate backbone (having no bases), when each amino acid is represented by a combination of the 1st and 2nd bases, the maximum number of combinations can be represented by 2×2×2=8 possibilities even in the case where there are two possible combinations, which is extremely advantageous when performing analysis.

Accordingly, a nucleic acid array according to the present invention that can accomplish the above object is characterized in that the above-mentioned oligonucleotide has a sequence site that forms base pairs with at least two bases of the four bases (A, T, C, G or U) or has a sequence site that does not form a base pair with any of the four bases.

In the present invention, it is preferred that an oligonucleotide having a sequence such that the 3rd position of a triplet (codon) defining one amino acid is abasic is immobilized on a substrate.

Further, a method of estimating an amino acid mutation according to the present invention that can accomplish the above object is characterized in that the above nucleic acid array is used and all the cases of nucleic acid mutations that are not accompanied by an amino acid mutation are efficiently concentrated on oligonucleotides having a sequence that is abasic, thereby distinguishing between a nucleic acid sequence that is accompanied by amino acid mutation and a nucleic acid sequence that is not accompanied by amino acid mutation through hybridization.

According to the present invention, a nucleic acid array can be provided that can perform analysis efficiently, and further, can estimate the number of all cases of amino acid mutation by use of a smaller number of arrays.

Other features and advantages of the present invention will be apparent from the following description taken in conjunction with the accompanying drawing.

### BRIEF DESCRIPTION OF THE DRAWINGS

The accompanying drawing, which is incorporated in and constitutes a part of the specification, illustrates an example of the invention and, together with the description, serves to explain the principles of the invention.

FIG. 1 is a schematic view illustrating a portion of a DNA microarray according to an embodiment of the present invention.

### BEST MODE FOR CARRYING OUT THE INVENTION

Preferred embodiments of the present invention will now be described in detail in accordance with the accompanying drawing.

The nucleic acid array according to the present invention is characterized by having a configuration such that two or more oligonucleotides having different base sequences are immobilized on a substrate, wherein each oligonucleotide has a sequence site that forms base pairs with at least two bases of the four bases (A, T, C, G or U) or has a sequence site that does not form a base pair with any of the four bases.

That is, a configuration such that there are only sequence moieties that is capable of forming a base-pair with any one base of the four bases (A, T, C, G or U) and incapable of forming a base pair with any of the remaining three bases is excluded from the above expression.

In particular, it is preferable that the oligonucleotide has a sequence site that does not perform selective base-pair formation with any of the above four bases. An example of such a kind of sequence site is an abasic site. Further, a sequence site that forms base pairs with all the four bases is also preferable. As an example of this type of sequence site, one having at least one site with a base selected from inosine, 5-nitroindole and 3-nitropyrrole may be included.

The nucleic acid array according to the present invention can be appropriately used as a microarray in which, for example, plural kinds of oligonucleotides are sectioned into individual oligonucleotides, for example, as a spot of each oligonucleotide, and disposed very densely in a desired arrangement.

Further, the sequence site may be one that is capable of forming base-pairs with any two bases of the above four bases (A, T, C, G or U).

As examples of this type of sequence site, there may be included one having P-imino (imino tautomers) or P-amino (amino tautomers) that is capable of forming a base-pair with A or G in an oligonucleotide, or one having K-imino (imino tautomers) or K-amino (amino tautomers) that is capable of forming a base-pair with C or T.

By allowing each oligonucleotide to function as a probe, the nucleic acid array according to the present invention can be utilized for detecting a specific DNA sequence or RNA sequence, or for detecting a mutation in a known DNA sequence or RNA sequence.

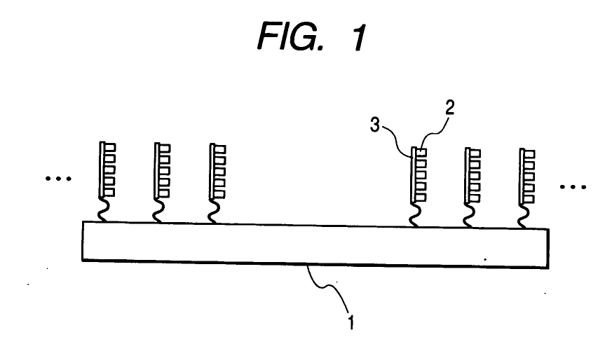

Hereafter, a case in which the present invention is applied to a DNA microarray is described. FIG. 1 is a schematic illustration of the structure of a portion of a DNA microarray that is an embodiment of the present invention. In the figure, a plurality of oligonucleotide spots is formed on a support member 1.

Any type of material may be employed for the support member 1, as long as the material is one that forms a solid phase surface for probe DNA or RNA, including glass, organic polymeric material such as plastic or the like. Further, the form of the support member 1 may be appropriately adapted to accord with a scanning device. For example, for a microplate reader the support member 1 may be well-shaped, for a microarray scanner the support member 1 may have a plate form, and for a device such as a flow cytometer the support member 1 may be in granular form.

The DNA array is one wherein oligonucleotides each having an abasic site are immobilized on the support member 1.

As an example of an abasic site, the 3rd base of a triplet encoding an amino acid may be mentioned. Herein, as a sugar for an abasic site, deoxyribose and ribose can be appropriately used in the case of DNA and RNA, respectively. For example, deoxyribose or ribose constituting a nucleoside can be used as such.

Hereunder, a DNA microarray having an abasic unit in the 3rd position is further explained.

As shown in FIG. 1, an oligonucleotide having an abasic site 2 that is lacking a base is immobilized to a sugar-phosphate backbone 3. Table 1 shows the relationship between amino acids and their corresponding codons. A case will be explained where, for example, a polypeptide having an amino acid sequence “Gly Ile Val Glu” is extracted from an mRNA sample and is used as a target. As an example of immobilizing an oligonucleotide having the 3rd base for an amino acid code replaced with an abasic unit, taking into consideration up to the second character of the codons shown in Table 1, the complementary strand of “GG-AT-GT-GA,” namely “CC-TA-CA-CT” (the base lacking site is represented with the symbol “-”), is immobilized on a substrate as a probe. For the base sequence coding for the above amino acid sequence when each amino acid is represented by a combination of three bases, theoretically there is a total of 4×3×4×2=96 possibilities. In contrast, by deleting the 3rd base according to the present invention, the base sequence coding for the above amino acid sequence can be determined from a total of just 2×2×2×2=16 possibilities. More specifically, by immobilizing oligonucleotides having a total of 16 different base sequences at pre-established locations on a substrate, it is possible to determine the base sequence that codes for the above amino acid sequence.

TABLE 1

| | | | |
|---|---|---|---|
| Amino acid | Codon | Amino acid | Codon |

| | | | |
|---|---|---|---|
| Alanine | GCA, GCC, | Lysine | AAA, AAG |
| | GCG, GCT | | |
| Arginine | CGA, CGC, | Methionine | ATG |
| | CGG, CGT, | | |
| | AGA, AGG | (initiation codon) | |
| Aspartic acid | GAC, GAT | Phenylalanine | TTC, TTT |
| Asparagine | AAC, AAT | Proline | CCA, CCC, |
| | | | CCG, CCT |
| Cysteine | TGC, TGT | Serine | TCA, TCC, |
| | | | TCG, TCT, |
| | | | AGC, AGT |
| Glutamic acid | GAA, GAG | Threonine | ACT, ACC, |
| | | | ACG, ACT |
| Glutamine | CAA, CAG | Tryptophan | TGG |
| Glycine | GGA, GGC, | Tyrosine | TAC, TAT |
| | GGG, GGT | | |
| Histidine | CAC, CAT | Valine | GTA, GTC, |
| | | | GTG, GTT |
| Isoleucine | ATA, ATC, | Termination codon | TAG, TAA, |
| | ATT | | TGA |
| Leucine | CTA, CTC, | | |
| | CTG, CTT, | | |
| | TTA, TTG | | |

The term “oligonucleotide” as herein employed is intended to embrace not only a deoxyribonucleotide polymer, a ribonucleotide polymer, and a polymer that contains both deoxyribonucleotides and ribonucleotides but also a compound in which some or all of the nucleotides as constitutional components thereof are replaced by nucleotide analogs.

Further, the nucleic acid part may be an LNA (Locked Nucleic Acid), which is said to have a high capability of identifying mismatchings.

The LNA is described in Proc. of Natl. Acad. of Sciences of the USA (Proc. Natl. Acad. Sci. USA), vol. 97, pp. 5633-5638 (2000).

Further, a peptide nucleic acid (PNA) bound by a peptide backbone instead of a sugar-phosphate ester backbone may be used.

The PNA is described in Nature, vol. 365, pp. 566-568 (1993), and the constitutional unit thereof is N-(2-aminoethyl)glycine to which bases are bound via a methylene carbonyl linker.

The term “nucleotide analog” as herein employed refers to a unit that can form a Watson-Crick base pair when some or all of nucleotides in a nucleic acid is substituted therewith. The nucleotide analog is not particularly limited, and a compound in which a sugar part or sugar-phosphate skeleton of a natural nucleotide is replaced by a group of a different structure can be appropriately used.

Those compounds containing a cross-linked ring structure as with the LNA or having a sugar part and a phosphate group replaced by an amino acid derivative as with the PNA may appropriately be used.

Any method can be used as a method for immobilizing the probe, and for example the method described in Japanese Patent Application Laid-Open No. H11-187900 may be used.

### EXAMPLES

Hereunder, the present invention will be specifically described with reference to examples, however the present invention is not limited to the following examples.

The present invention will be specifically explained referring to the protein sequence of HLA-DRA acquired from the database of the NCBI (National Centre for Biotechnology Information, Bethesda, Md., USA).

TABLE 2

| | |
|---|---|
| LOCUS | HLHUDA 254 aa PRI 22-JUN-1999 |
| DEFINITION | MHC class II histocompatibility |
| | antigen HLA-DR alpha chain |
| | precursor-human. |
| ACCESSION | HLHUDA |
| PID | g70099 |
| VERSION | HLHUDA GI: 70099 |
| PROTEIN SEQUENCE- | 254 aa |
| | MAISGVPVLGFFIIAVLMSAQESWAIKEEHVI |
| | IQAEFYLNPDQSGEFMFDFDGDEIFHVDMAKK |
| | ETVWRLEEFGRFASFEAQGALANIAVDKANLE |
| | IMTKRSNYTPITNVPPEVTVLTNSPVELREPN |
| | VLICFIDKFTPPVVNVTWLRNGKPVTTGVSET |
| | VFLPREDHLFRKFHYLPFLPSTEDVYDCRVEH |
| | WGLDEPLLKHWEFDAPSPLPETTENVVCALGL |
| | TVGLVGIIIGTIFIIKGVRKSNAAERRGPL |

The above sequence was sectioned from the beginning by a unit size of 12 amino acids and converted into a probe array with a total of 21 skeletons. In the skeletons, even when the 3rd character of the amino acid codon is rendered abasic, there still exist those having two possibilities. It is known that those are R (arginine), L (leucine), S (serine), Y (tyrosine) and C (cysteine). Table 3 shows the sectionalized 21 skeletons according to the present example. Further, Table 3 also shows the number of probes necessary to detect a base sequence coding for each skeleton when the 3rd character of the amino acid codons is rendered abasic. In Table 3, no description is provided where only one possibility exists.

TABLE 3

Skeleton

| | |
|---|---|
| MAISGVPVLGFF | × 4 possibilities |
| IIAVLMSAQESW | × 8 possibilities |
| AIKEEHVIIQAE | |
| FYLNPDQSGEFM | × 8 possibilities |
| FDFDGDEIFHVD | |
| MAKKETVWRLEE | × 4 possibilities |
| FGRFASFEAQGA | × 4 possibilities |
| LANIAVDKANLE | × 4 possibilities |
| IMTKRSNYTPIT | × 8 possibilities |
| NVPPEVTVLTNS | × 4 possibilities |
| PVELREPNVLIC | × 16 possibilities |
| FIDKFTPPVVNV | |
| TWLRNGKPVTTG | × 4 possibilities |
| VSETVFLPREDH | × 8 possibilities |
| LFRKFHYLPFLP | × 32 possibilities |
| STEDVYDCRVEH | × 16 possibilities |
| WGLDEPLLKHWE | × 4 possibilities |
| FDAPSPLPETTE | × 4 possibilities |
| NVVCALGLTVGL | × 16 possibilities |
| VGIIIGTIFIIKG | |
| VRKSNAAERRGPL | × 32 possibilities |

For example, if four of those having two possibilities mentioned above are contained in a probe array necessary to detect a base sequence of one skeleton, a total of 2×2×2×2=16 possibilities exists. Therefore, an array having probes of the same number as the total number of the possibilities for that skeleton (i.e., 16 possibilities) was made, and the same procedure was carried out for all the skeletons to prepare a DNA array having a total of 180 kinds of probes of different given sequences immobilized on a substrate. Incidentally, in order to align the Tm values of the respective probes with respect to the sequence of the basic skeleton as far as possible, there may be carried out such adjustment as to extend or reduce the bases at both ends by several units.

Hereunder, the present invention will be explained in more detail using examples.

### EXAMPLE 1

The total of 180 probes described above having the 3rd base of codons substituted with an abasic unit were all acquired from a synthetic oligonucleotide manufacturer as oligonucleotides having an SH group introduced at the terminal on the solid phase side. In the present example, the immobilization of probes was performed according to the example described in Japanese Patent Application Laid-Open No. H11-187900, to produce a microarray having 180 spots disposed on a glass substrate.

Since MHC-DRA migrates to substantially the same location for all humans at the separation level of electrophoresis, it is not clear whether a mutation occurs at the amino acid level. However, by producing a DNA chip of the abasic probe array of the present example, it is possible to estimate the number of amino acid mutations for all cases based on differences in fluorescence intensity when a mismatching occurs during hybridization, using a smaller number of arrays.

### EXAMPLE 2

In the present example, instead of using oligonucleotides having abasic sites, oligonucleotides having inosine introduced into the abasic sites of Example 1 were used. Further, the total of 180 oligonucleotides having an SH group introduced at the terminal was acquired from a synthetic oligonucleotide manufacturer.

An oligonucleotide microarray was produced following the same procedure as in Example 1. Since the level of fluorescence intensity obtained after a hybridization reaction was higher than the value obtained for the microarray of Example 1, it was seen that the stability of hybrids during hybridization was higher for the microarray of the present example.

### EXAMPLE 3

In the present example, a microarray was produced following the same procedure as in Example 2 l with the exception that the nucleic acid part of the oligonucleotides used in Example 2 was replaced by LNA. Since the level of fluorescence intensity obtained after a hybridization reaction was even higher than the value for the microarray of Example 2, it was seen that the stability of hybrids during hybridization was further higher for the microarray of the present example.

### EXAMPLE 4

For the probes according to Example 1, probe representations were designated as shown in the following table with respect to the amino acids listed below.

Probe

| | | |
|---|---|---|
| Amino acid | Codon | representation |

| | | |
|---|---|---|
| Arginine | CGA, CGC, CGG, | CGN or AGK |
| | CGT, AGA, AGG | |
| Aspartic acid | GAC, GAT | GAP |
| Asparagine | AAC, AAT | AAP |
| Cysteine | TGC, TGT | TGP |
| Glutamic acid | GAA, GAG | GAK |
| Glutamine | CAA, CAG | CAK |
| Histidine | CAC, CAT | CAP |
| Leucine | CTA, CTC, CTG, | CTN or TTK |
| | CTT, TTA, TTG | |
| Lysine | AAA, AAG | AAK |
| Phenylalanine | TTC, TTT | TTP |
| Serine | TCA, TCC, TCG, | TCN or AGP |
| | TCT, AGC, AGT | |
| Tryptophan | TGG | TGG |
| Tyrosine | TAC, TAT | TAP |

In the above column showing probe representation, P represents P-imino, K represents K-imino, and N represents 5-Nitroindole. For amino acids other than those shown in the above table, a microarray was produced according to the method described in Example 1. When DNA was extracted from blood of a human and a pig, and hybridization was conducted for this microarray and the microarray of Example 2, it was seen that the specificity of fluorescence intensity of this microarray was slightly superior to that of the microarray of Example 2.

As described above, according to the present invention, by using a nucleic acid array in which oligonucleotides having a sequence site that forms base pairs with at least two bases of the four bases (A, T, C, G or U) or oligonucleotides having a sequence site that does not form a base pair with any of the four bases are immobilized on a substrate, analysis can be efficiently conducted, and further, it is possible to estimate the number of expressions of amino acid mutations for all cases by use of a smaller number of arrays. In addition, by using the DNA probe array of the present invention, a method can be provided for efficiently estimating, with a smaller number of arrays, the number of expressions of mismatches for all cases for amino acid mutations at the time of occurrence of mismatching during hybridization.

The present invention is not limited to the above embodiments and various changes and modifications can be made within the spirit and scope of the present invention. Therefore to apprise the public of the scope of the present invention, the following claims are made.

1. A nucleic acid array having a plurality of oligonucleotides immobilized on a substrate, wherein the oligonucleotide has a sequence site that forms base pairs with at least two bases of the four bases (A, T, C, G or U) or has a sequence site that does not form a base pair with any of the four bases.

2. The nucleic acid array according to claim 1, wherein the sequence site is a sequence site of a unit comprising a backbone having no base or a sequence site having a base selected from the group consisting of inosine, P-amino, P-imino, K-amino, K-imino, 5-Nitroindole, and 3-Nitropyrrole.

3. The nucleic acid array according to claim 1, wherein the sequence site is a sequence site of a unit comprising a backbone having no base.

4. The oligonucleotide array according to claim 1, wherein in a partial sequence of the oligonucleotide, successive triplets correspond to codons of amino acids and a third base of the codon forms the sequence site.

5. A detection method comprising detecting a target nucleic acid using the nucleic acid array set forth in claim 1.

6. A method of distinguishing an amino acid mutated sequence, comprising distinguishing between a nucleic acid mutated sequence not accompanied by an amino acid mutation and a nucleic acid mutated sequence accompanied by an amino acid mutation through hybridization, using the nucleic acid array set forth in claim 1.


SEQUENCE LISTING (1 sequence)

SEQ ID NO: 1 - protein, 254 aa, Homo sapiens
MAISGVPVLGFFIIAVLMSAQESWAIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTENVVCALGLTVGLVGIIIGTIFIIKGVRKSNAAERRGPL